(19) United States
(12) Patent Application Publication
(10) Pub. No.: US 2007/0238974 A1
(43) Pub. Date: Oct. 11, 2007

(54) THREE-DIMENSIONAL MAGNETIZATION PREPARED MRI ACQUISITION WITH INCREASED ACQUISITION WINDOW

(76) Inventors: Chen Lin, Westfield, IN (US); Matthew A. Bernstein, Rochester, MN (US)

Correspondence Address: QUARLES & BRADY LLP, 411 E. WISCONSIN AVENUE, SUITE 2040, MILWAUKEE, WI, 53202-4497, US

(21) Appl. No.: 11/697,380

(22) Filed: Apr. 6, 2007

Related U.S. Application Data
(60) Provisional application No. 60/790,040, filed on Apr. 7, 2006.

(51) Int. Cl. G01V 3/00; A61B 5/05

(52) U.S. Cl. 600/410; 324/309

(57) ABSTRACT

A magnetization preparation pulse is followed by acquiring a segment of k-space data during an acquisition window in which a desired tissue contrast is achieved. Views sampling the center of k-space are acquired at peak contrast and peripheral k-space is sampled before and after this optimal contrast time.

### CROSS-REFERENCE TO RELATED APPLICATION

This application claims the benefit of U.S. provisional patent application Ser. No. 60/790,040 filed on Apr. 7, 2006 and entitled “Three-Dimensional Prepared Elliptical Centric Fast Gradient Echo Magnetic Resonance Imaging.”

### BACKGROUND OF THE INVENTION

The field of the invention is nuclear magnetic resonance imaging (MRI) methods and systems. More particularly, the invention relates to magnetization prepared MRI.

When a substance such as human tissue is subjected to a uniform magnetic field (polarizing field B₀), the individual magnetic moments of the spins in the tissue attempt to align with this polarizing field, but precess about it in random order at their characteristic Larmor frequency. If the substance, or tissue, is subjected to a magnetic field (excitation field B₁) which is in the x-y plane and which is near the Larmor frequency, the net aligned longitudinal magnetization, M_{z}, may be rotated, or “tipped”, into the x-y plane to produce a net transverse magnetization Mₜ. A signal is emitted by the excited spins after the excitation signal B₁ is terminated, and this signal may be received and processed to form an image.

When utilizing these signals to produce images, magnetic field gradients (Gₓ, G_{y} and G_{z}) are employed. Typically, the region to be imaged is scanned by a set of measurement cycles, or “views”, in which these gradients vary according to the particular localization method being used to sample different parts of k-space. The resulting set of received NMR signals are digitized and processed to reconstruct the image using one of many well known reconstruction techniques.

Magnetization preparation is a method used to enhance the contrast between tissue types in an MR image. As shown in FIG. 3, for example, when imaging the brain it is often desirable to increase the contrast between white matter and gray matter by applying a magnetization preparation RF pulse such as an inversion pulse 50 before performing a measurement cycle 52 to acquire one or more NMR signals. Usually we reconstruct and display a magnitude image, so that the image intensity is proportional to the absolute value of the longitudinal magnetization just before the RF excitation pulse. This is the case depicted in FIG. 3, where the magnetization preparation RF pulse 50 inverts the magnetization; T1—recovery causes the absolute value of the signal from white matter plotted versus T1—to drop to zero and then increase as indicated by dotted line 54; and it causes the signals from gray matter to drop and recover as indicated by dashed line 56. The difference in tissue contrast in an image acquired at different times TI after the magnetization preparation (MP) pulse 50 is indicated by solid line 58 and the goal is to perform the measurement pulse sequence when this difference in contrast is at a high value. If only a single pulse sequence measurement cycle 52 is performed after each MP pulse, considerable dead time TD results while the longitudinal magnetization recovers and another cycle is repeated. Many repetitions of the pulse sequence measurement cycle are required to sample sufficient k-space data to reconstruct a 3D image and the total scan time becomes very lengthy.

A phase sensitive or real reconstruction can be used instead of a magnitude reconstruction, as taught in Xiang Q S, Inversion Recovery Image Reconstruction With Multiseed Region-Growing Spin Reversal, J. Magn. Reson. Imaging, September-October 1996; 6(5):775-82. This reconstruction method for the inversion-recovery magnetization prepared image can increase the dynamic range because both positive and negative pixel values are displayed. If a phase-sensitive reconstruction is used instead of a magnitude reconstruction, then the dotted and dashed plots in FIG. 3 can each take on negative values and the solid line can display increased contrast.

Referring still to FIG. 3, one solution to this problem is to acquire a segment 60 of NMR data that contains more than one view during a larger acquisition window while the contrast is at a maximum. Well known methods such as MP-RAGE and IR-SPGR described by Liang L et al, AJNR 2002; 23:1739-1749; Green J D et al, JMRI 2002; 16:104-109; and Held P et al EJR 2001; 37:18-25 and variations thereof described by Deichmann R et al, Neuromage 2000; 12-112-127; Bampton A H et al, JMRI 1992; 2:237-334; Rofsky N M et al, Radiology 1999; 212:876-884; Wetzel S et al, AJNR 2002 23(6):995-1002 use this technique to shorten scan time. While these prior methods reduce the deadtime TD and shorten the overall scan time, the scans are still too long and further improvement is sought.

### SUMMARY OF THE INVENTION

The present invention is a magnetization preparation method for acquiring MRI data in which the size of the acquisition window is increased to shorten scan time without decreasing image contrast. This is achieved by ordering the views that are acquired during each acquisition window such that the center of k-space is sampled during the portion of the window in which contrast preparation is optimal and sampling peripheral k-space during less than optimal conditions. Because image contrast is dominated by samples acquired at the center of k-space, it has been discovered that peripheral k-space can be sampled at less than optimal contrast conditions without substantially impacting image contrast. This enables the acquisition window to be increased in size as indicated at 62 in FIG. 3 with a consequent reduction in dead time TD and total scan time.

Instead of sampling all the data points, or views, in the ky-kz plane, only the views that sample k-space within an ellipsoid are acquired, as shown in FIG. 2. Usually, this ellipsoid is inscribed by the standard ky-kz rectangle to maintain the image resolution. The views to be acquired are divided into segments so that one segment of views is acquired after each magnetization preparation rf pulse 50 is produced. The acquisition order of views in each segment is based on the distance of the k-space sampling from the center of k-space, or their k-space radii. Considering that the contrast to noise ratio (CNR) between two tissue types with different relaxation times peaks at a different time than the signal to noise ratio (SNR) after magnetization preparation, the central views of k-space are acquired during a narrow window when CNR is peak, while the peripheral views of k-space are acquired at different times during the acquisition window 62 to improve the SNR and data acquisition efficiency.

### BRIEF DESCRIPTION OF THE DRAWINGS

FIG. 1 is a block diagram of an MRI system which employs the present invention.

FIG. 2 is a pictorial view of k-space showing the region that is sampled during a scan;

FIG. 3 is a graphic view of a magnetization preparation sequence showing the data acquisition window as practiced by the prior art and as practiced using the present invention;

FIG. 4 is a graphic view of an exemplary three-dimensional MR imaging pulse sequence that may be used with the present invention;

FIG. 5 is a graphic view of a magnetization preparation sequence according to the present invention; and

FIG. 6 is a pictorial view of the k-space sampling pattern used in the sequence of FIG. 5.

### DESCRIPTION OF THE PREFERRED EMBODIMENT

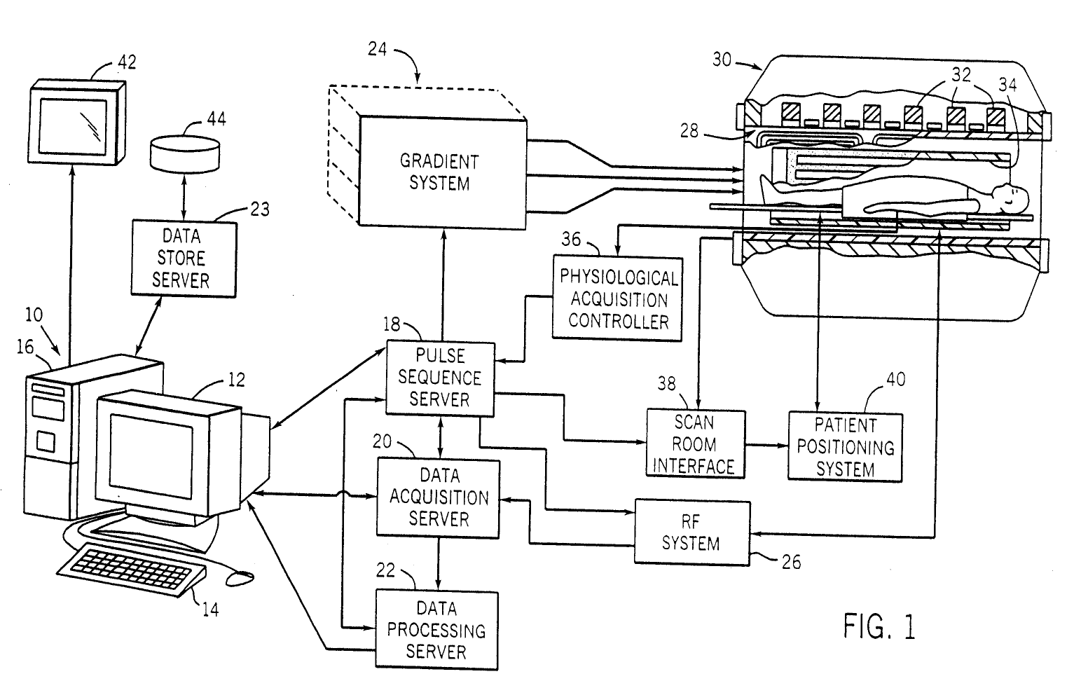

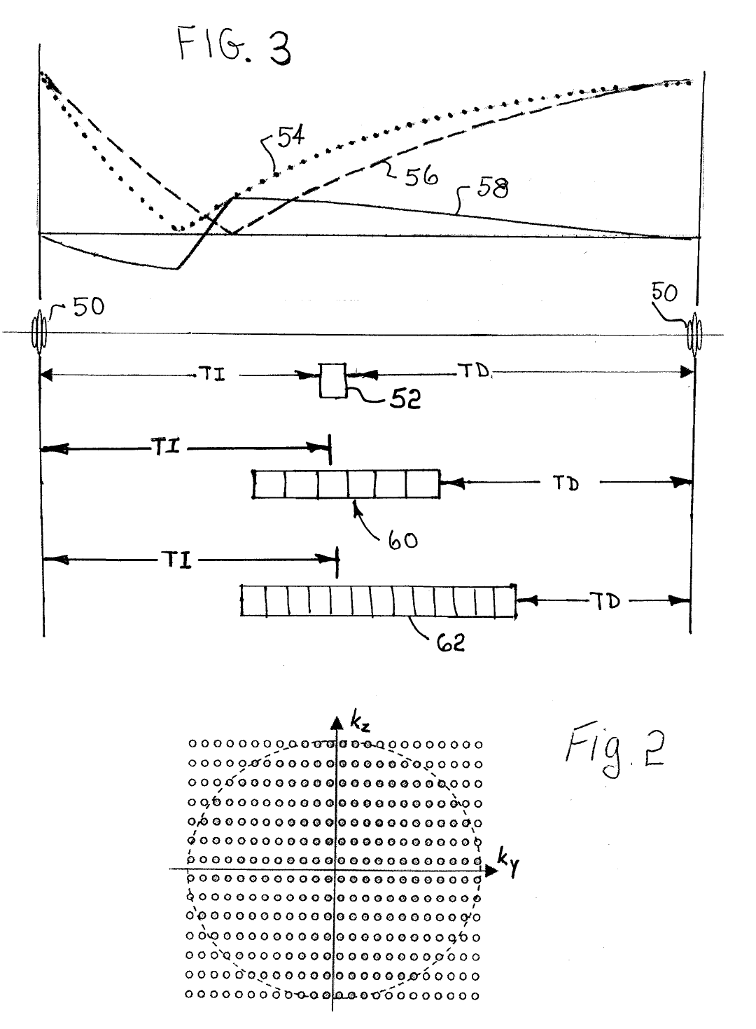

Referring particularly to FIG. 1, the preferred embodiment of the invention is employed in an MRI system. The MRI system includes a workstation 10 having a display 12 and a keyboard 14. The workstation 10 includes a processor 16 which is a commercially available programmable machine running a commercially available operating system. The workstation 10 provides the operator interface which enables scan prescriptions to be entered into the MRI system.

The workstation 10 is coupled to four servers: a pulse sequence server 18; a data acquisition server 20; a data processing server 22, and a data store server 23. In the preferred embodiment the data store server 23 is performed by the workstation processor 16 and associated disc drive interface circuitry. The remaining three servers 18, 20 and 22 are performed by separate processors mounted in a single enclosure and interconnected using a 64-bit backplane bus. The pulse sequence server 18 employs a commercially available microprocessor and a commercially available quad communication controller. The data acquisition server 20 and data processing server 22 both employ the same commercially available microprocessor and the data processing server 22 further includes one or more array processors based on commercially available parallel vector processors.

The workstation 10 and each processor for the servers 18, 20 and 22 are connected to a serial communications network. This serial network conveys data that is downloaded to the servers 18, 20 and 22 from the workstation 10 and it conveys tag data that is communicated between the servers and between the workstation and the servers. In addition, a high speed data link is provided between the data processing server 22 and the workstation 10 in order to convey image data to the data store server 23.

The pulse sequence server 18 functions in response to program elements downloaded from the workstation 10 to operate a gradient system 24 and an RF system 26. Gradient waveforms necessary to perform the prescribed scan are produced and applied to the gradient system 24 which excites gradient coils in an assembly 28 to produce the magnetic field gradients Gₓ, G_{y} and G_{z} used for position encoding NMR signals. The gradient coil assembly 28 forms part of a magnet assembly 30 which includes a polarizing magnet 32 and a whole-body RF coil 34.

RF excitation waveforms are applied to the RF coil 34 by the RF system 26 to perform the prescribed magnetic resonance pulse sequence. Responsive NMR signals detected by the RF coil 34 are received by the RF system 26, amplified, demodulated, filtered and digitized under direction of commands produced by the pulse sequence server 18. The RF system 26 includes an RF transmitter for producing a wide variety of RF pulses used in MR pulse sequences. The RF transmitter is responsive to the scan prescription and direction from the pulse sequence server 18 to produce RF pulses of the desired frequency, phase and pulse amplitude waveform. The generated RF pulses may be applied to the whole body RF coil 34 or to one or more local coils or coil arrays.

The RF system 26 also includes one or more RF receiver channels. Each RF receiver channel includes an RF amplifier that amplifies the NMR signal received by the coil to which it is connected and a quadrature detector which detects and digitizes the I and Q quadrature components of the received NMR signal. The magnitude of the received NMR signal may thus be determined at any sampled point by the square root of the sum of the squares of the I and Q components:

M=\sqrt{I²+Q²},

and the phase of the received NMR signal may also be determined:

φ=tan⁻¹ Q/I.

The pulse sequence server 18 also optionally receives patient data from a physiological acquisition controller 36. The controller 36 receives signals from a number of different sensors connected to the patient, such as ECG signals from electrodes or respiratory signals from a bellows. Such signals are typically used by the pulse sequence server 18 to synchronize, or “gate”, the performance of the scan with the subject's respiration or heart beat.

The pulse sequence server 18 also connects to a scan room interface circuit 38 which receives signals from various sensors associated with the condition of the patient and the magnet system. It is also through the scan room interface circuit 38 that a patient positioning system 40 receives commands to move the patient to desired positions during the scan.

It should be apparent that the pulse sequence server 18 performs real-time control of MRI system elements during a scan. As a result, it is necessary that its hardware elements be operated with program instructions that are executed in a timely manner by run-time programs. The description components for a scan prescription are downloaded from the workstation 10 in the form of objects. The pulse sequence server 18 contains programs which receive these objects and converts them to objects that are employed by the run-time programs.

The digitized NMR signal samples produced by the RF system 26 are received by the data acquisition server 20. The data acquisition server 20 operates in response to description components downloaded from the workstation 10 to receive the real-time NMR data and provide buffer storage such that no data is lost by data overrun. In some scans the data acquisition server 20 does little more than pass the acquired NMR data to the data processor server 22. However, in scans which require information derived from acquired NMR data to control the further performance of the scan, the data acquisition server 20 is programmed to produce such information and convey it to the pulse sequence server 18. For example, during prescans NMR data is acquired and used to calibrate the pulse sequence performed by the pulse sequence server 18. Also, navigator signals may be acquired during a scan and used to adjust RF or gradient system operating parameters or to control the view order in which k-space is sampled. And, the data acquisition server 20 may be employed to process NMR signals used to detect the arrival of contrast agent in an MRA scan. In all these examples the data acquisition server 20 acquires NMR data and processes it in real-time to produce information which is used to control the scan.

The data processing server 22 receives NMR data from the data acquisition server 20 and processes it in accordance with description components downloaded from the workstation 10. Such processing may include, for example: Fourier transformation of raw k-space NMR data to produce two or three-dimensional images; the application of filters to a reconstructed image; the performance of a backprojection image reconstruction of acquired NMR data; the calculation of functional MR images; the calculation of motion or flow images, etc.

Images reconstructed by the data processing server 22 are conveyed back to the workstation 10 where they are stored. Real-time images are stored in a data base memory cache (not shown) from which they may be output to operator display 12 or a display 42 which is located near the magnet assembly 30 for use by attending physicians. Batch mode images or selected real time images are stored in a host database on disc storage 44. When such images have been reconstructed and transferred to storage, the data processing server 22 notifies the data store server 23 on the workstation 10. The workstation 10 may be used by an operator to archive the images, produce films, or send the images via a network to other facilities.

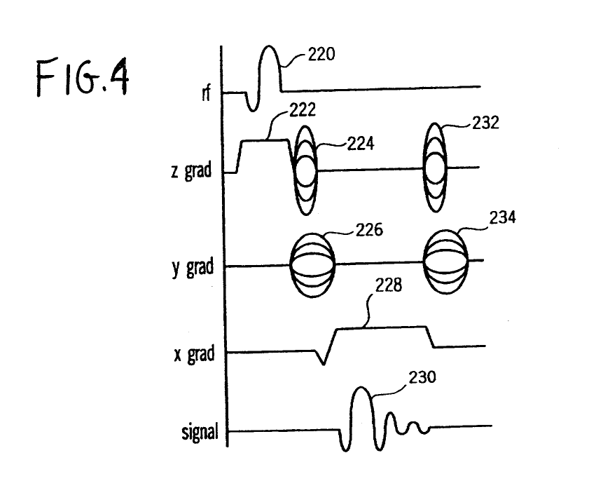

Referring to FIG. 4, while many different pulse sequences can be used to acquire k-space samples from the ellipsoid region in FIG. 2, in the preferred embodiment a 3D gradient-recalled echo pulse sequence is employed. It includes an RF excitation pulse 220 having a flip angle of 8° is produced in the presence of a slab select gradient pulse 222 to produce transverse magnetization in the 3D volume of interest as taught in U.S. Pat. No. 4,431,968. This is followed by a phase encoding gradient pulse 224 directed along the z axis and a phase encoding gradient pulse 226 directed along the y axis. A readout gradient pulse 228 directed along the x axis follows and a partial echo (60%) NMR signal 230 is acquired and digitized as described above. After the acquisition, rewinder gradient pulses 232 and 234 rephase the magnetization before the pulse sequence is repeated as taught in U.S. Pat. No. 4,665,365 and a spoiler gradient is applied along one or more of the gradient axes to dephase any residual transverse magnetization.

As is well known in the art, the pulse sequence is repeated and the phase encoding pulses 224 and 226 are stepped through a series of values to sample along k_{z} and k_{y} in 3D k-space. As will become apparent from the discussion below, the order in which this sampling is performed is an important aspect of the present invention.

Sampling along the kₓ axis is performed by sampling the echo signal 230 in the presence of the readout gradient pulse 228 during each pulse sequence. It will be understood by those skilled in the art that only a partial sampling along the kₓ axis is performed and the missing data is computed using a homodyne reconstruction or by zero filing. This enables the echo time (TE) of the pulse sequence to be shortened to less than 1.8 to 2.0 ms. and the pulse repetition rate (TR) to be shortened to less than 10.0 msecs.

Each repetition of the imaging pulse sequence of FIG. 4 samples k-space along the readout axis at one selected location, or “view”, in k_{y}/k_{z} space. This pulse sequence is repeated many times to sample throughout the ellipsoid region of k_{y}/k_{z} space and it is the order and timing in which this is done that is the subject of the present invention.

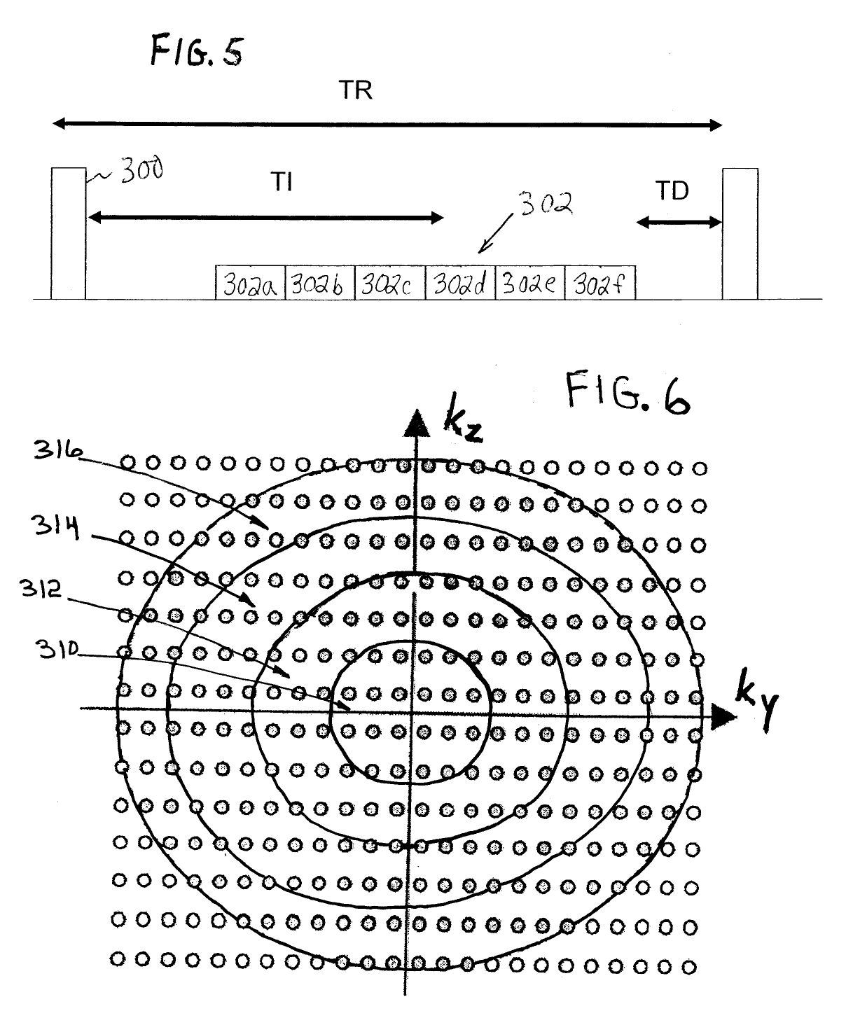

Referring particularly to FIG. 5, to enhance tissue contrast the pulse sequence is acquired after the spin magnetization has been prepared by an rf inversion pulse 300. The tissue contrast produced by the inversion pulse 300 changes over time and during a period in which the desired contrast is at a high level, the pulse sequence is repeated as indicated at 302 to acquire as many “views” as possible. It is a teaching of the present invention that the order and timing of the views that are acquired is important. More particularly, at a time TI after each inversion pulse 300 is produced and the desired tissue contrast is at a maximum, the pulse sequence that is performed samples from the center of k_{y}-k_{z} space. In general, the center of k-space is sampled closer to this time TI and more peripheral k-space is sampled further from this TI time, either before or after.

There are many acquisition orders that may be employed to practice the present invention. As shown in FIG. 6, in the preferred embodiment the k_{z}/k_{y} space to be sampled is divided into annular regions, including a central region 310 and three peripheral k-space regions 312, 314 and 316. As shown in FIG. 5, six views are acquired during each segment 302 and views are ordered such that the first view 302a acquires peripheral k-space data in region 316, the second view 302b acquires data from region 314, the third view 302c is acquired in peripheral region 312, and the view 302d performed at the optimal contrast time TI samples the central region 310 of k-space. This is sometimes referred to as a descending view order. The last two views 302e and 302f are then acquired in ascending order, with view 302e sampling peripheral region 312 and view 302f sampling peripheral region 314. The number of views acquired in each segment 302 will of course vary depending on the clinical application, as will the number of separate peripheral regions that are established.

Scans were conducted using an 8-channel head coil with both MP-RAGE and MP-EFGRE that employs the present invention for direct comparison. Imaging parameters optimized by the ADNI study described by Loew A D et al, Longitudinal Stability of MRI for Mapping Grain Change Using Tensor-Based Morphometry, Neuroimage, June 2006; 31(2):627-40, were used to acquire MP-RAGE and MP-EFGRE images with 26 cm FOV, 256×256 matrix, 0.94 phase FOV, 1.2 mm slice thickness, 170 slices, 8 deg flip angle, 31.25 kHz BW, 900 ms TI, and 2300 ms TR. However, the number of views in each segment were adjusted in MP-EFGRE acquisition to achieve various compromises between CNR, SNR and scan time.

Table 1 shows the comparison of CNR, SNR and scan time between MP-EFGRE and MP-RAGE. As the number of views per segment in the invented MP-EFGRE acquisition increases, the delay TD between the acquisition of data and the next magnetization preparation section is reduced.

TABLE 1
Comparison of image contrast-to-noise ratio (CNR),
signal-to-noise(SNR) and scan time between the existing
MP-RAGE method and the new MP-EFGRE method
with a different number of views per segment.

| Type | Views per segment | CNR | SNR | Time |
|---|---|---|---|---|
| MP-RAGE | 170 | 6.5 | 18.7 | 9:14 |
| MP-EFGRE | 170 | 7.1 | 19.6 | 7:16 |
| MP-EFGRE | 190 | 6.8 | 19.9 | 6:30 |
| MP-EFGRE | 230 | 6.0 | 21.1 | 5:23 |

Because the k-space data points are sampled in a pseudo-random fashion instead of line by line as in other methods, MP-EFGRE effectively disperses structured artifacts as pseudo noise.

With the same number of views per segment as in MP-RAGE, MP-EFGRE images have better image quality in terms of CNR, SNR, less artifacts and shorter scan time. A further increase of SNR and reduction of scan time can be achieved with additional views per segment. Although the CNR started to decrease, it was still higher than the MP-RAGE method.

In the preferred embodiment described above magnitude images are constructed and the contrast between tissue types is based on the difference in magnitude of the signals produced by the tissues. It should be apparent to those skilled in the art that the present invention may also be employed with a phase sensitive image reconstruction where the contrast between tissue types is based on the phase of the signals produced by the tissues.

1. A method for producing a 3D image with a magnetic resonance imaging (MRI) system, the steps comprising:
a) producing an rf inversion pulse with the MRI system;
b) repeatedly performing a plurality of imaging pulse sequences to acquire a segment of k-space data during a period of time following step a) in which a desired tissue contrast is achieved, each imaging pulse sequence operating the MRI system to sample k-space along a k-space trajectory, and wherein the plurality of pulse sequences are ordered such that those which sample at selected k-space locations near the center of k-space are performed when tissue contrast is at a maximum;
c) repeating steps a) and b) to sample three-dimensional k-space throughout a selected region; and
d) reconstructing a 3D image from the acquired k-space samples.

2. The method s recited in claim 1 in which the selected region is an ellipsoid disposed around the center of k-space.

3. The method as recited in claim 1 in which each pulse sequence is a gradient-recalled echo pulse sequence.

4. The method as recited in claim 1 in which the respective image pulse sequences sample k-space in descending view order and then ascending view order.
